(19) United States
(12) Patent Application Publication
(10) Pub. No.: US 2009/0221890 A1
(43) Pub. Date: Sep. 3, 2009

(54) Diabetes Management System

(76) Inventors: Daniel Saffer, San Francisco, CA (US); Alexa Andrzejewski, San Francisco, CA (US); Rachel Hinman, San Francisco, CA (US); Sebastian Heycke, San Francisco, CA (US); Jamin Hegeman, Pittsburgh, PA (US)

Correspondence Address: MORGAN, LEWIS & BOCKIUS, LLP. (PA), 2 PALO ALTO SQUARE, 3000 EL CAMINO REAL, SUITE 700, PALO ALTO, CA, 94306, US

(21) Appl. No.: 12/039,722

(22) Filed: Feb. 28, 2008

(51) Int. Cl. A61B 5/05; A61M 1/00

(52) U.S. Cl. 600/347; 604/66; 604/67

(57) ABSTRACT

In one embodiment, a diabetes management system comprises a glucose monitoring system, a pump system, and a remote device. The glucose monitoring system and pump system are attached to a patient and covered by a soft shell. The glucose monitoring system and the pump system are controlled by the small, touch-screen remote device such a patient can discreetly monitor blood glucose levels and administer insulin dosages. The remote device has a small and durable form factor that can be worn or carried in various ways.

### RELATED APPLICATIONS

The present application relates to design application number ______, to Daniel Saffer et al., filed Feb. 28, 2008, entitled “Remote Control Device for a Diabetes Management System”, which also relates to design application number ______, to Daniel Saffer et al., also filed Feb. 28, 2008, entitled “Soft Shell for a Diabetes Management System”, the disclosures of which are hereby incorporated by reference in their entirety.

### FIELD OF THE INVENTION

The present invention relates generally to the field of medical devices, and in particular, to a diabetes management system that includes a pump, a glucose system, and a remote device for communicating with the pump and glucose monitor.

### BACKGROUND OF THE INVENTION

Patients with diabetes, otherwise known as diabetics, have a chronic disease that is characterized by a disordered metabolism resulting in high blood sugar. There are two types of diabetes. Type 1 diabetes involves a permanent loss or destruction of the beta cells of the pancreas which produces insulin. This often results in low levels or the complete absence of naturally produced insulin in the body. Type 2 diabetes involves a combination of an unusual resistance to naturally produced insulin and a relative insulin deficiency. As a result of both types of diabetes, a patient's blood sugar must be specially regulated. In the case of Type 2 diabetes, blood sugar regulation can sometimes be accomplished through a carefully maintained diet and exercise regime. In the case of Type 1 diabetes, blood sugar regulation almost always requires insulin supplementation.

Supplementation of insulin traditionally comes in two forms, basal injections and bolus injections. Basal injections are low level insulin injections meant to cover the patient's general insulin deficiency at low insulin need times such as between meals and at night, although even during higher insulin need times the basal injections may be administered. Bolus injections are higher level insulin injections usually administered before a meal or to correct an unusually high blood glucose level.

Both Type 1 and Type 2 diabetics who require insulin supplementation have a cycle that they repeat several times a day: checking their blood sugar level (otherwise known as blood glucose level), interpreting the results of that test, and then acting to adjust their blood sugar if necessary. A patient's blood glucose level is often checked using a finger prick mechanism, capturing the blood on a testing strip or feeding a certain amount of blood into a glucose level reader. After checking blood glucose levels, diabetics need to interpret the results of their blood glucose test. Traditionally, the blood glucose level reading is simply a raw number. Normally, blood glucose levels are between 70 to 150 mg/dL. If the patient's blood glucose level falls outside of this range, the patient will need to adjust it using an insulin injection. Generally, insulin dosages are calculated based on a formula that is unique to each patient, and then adjustments are made based on several other changing factors. These factors include taking into account what and how much food the patient had or will be having shortly, if the patient has just completed or plans to complete a specified amount of physical activity, and whether the patient has switched or plans to switch into a different mode of operation such as switching from awake mode to sleep mode. Once all of these factors have taken into account and an insulin dosage has been calculated, then the insulin is injected. Insulin is injected either with a syringe or a pump. This cycle must be done as often as necessary and is complex, time consuming, uncomfortable, and sometimes even embarrassing.

In order to perform these steps there are currently products on the market that help Diabetics take blood glucose readings, interpret results, and adjust blood glucose levels. However, the current products require diabetics carry-around with them and manage a tremendous amount of equipment. Diabetics often have to carry a glucose meter, a lancet for taking blood samples, and testing strips, an insulin syringe, one or more vials of insulin or an insulin pump. Taking a blood glucose reading manually requires a finger prick blood sample, catching the blood sample on a testing strip, and feeding the testing strip into the blood glucose meter. This is painful, potentially embarrassing, prone to error, and cannot be done while the patient is involved in other activities such as exercise or sleeping. Additionally, the equipment is often aesthetically unpleasing and “medical looking”; there are too many parts to easily manage; equipment is unsightly when seen poking out from under the patient's clothing; the equipment is bulky to carry in a shirt or pants pocket; the equipment is typically not waterproof; the long cannula stretching from the pump to the injection site can be uncomfortable and irritating for patient and can get caught on other objects; and the equipment comes in a limited choice of colors. Furthermore, current products are not context aware and they do not record and learn from the testing and dosing operations performed by the patient. Currently, there are no integrated products that help a patient easily test, interpret, and dose discretely.

Diabetes can be a very fatiguing disease to live with. Diabetes is a disease that must be constantly managed. Diabetes forces people to be more regimented in their lifestyles, eating patterns, and awareness of time. Furthermore, diabetes is typically managed alone, often without much community support. Remaining motivated to provide proper self care, especially over decades of living with the disease, is a significant challenge. Despite these serious problems, current products on the market assist very little in helping diabetics comfortably live with and manage their disease, set goals, or keep motivated.

Therefore, it would be highly desirable to provide a system and method for addressing the above mentioned problems associated with wearing and using diabetic devices, checking blood glucose, interpreting test results, adjusting blood glucose, and remaining motivated to deal with the disease. Specifically, it would be desirable to provide an integrated system that has fewer and smaller components; is easier to wear; can make better use of data; keeps patients in control; is easy to learn and teach; involves less interpretation by the patient (e.g., fewer numbers); and gives the patient a platform to view and share data, view long term trends, interact with a health care professional, and provide other methods for remaining motivated.

### SUMMARY

In some embodiments, a patient wears a glucose monitor and a pump system covered by a soft, flesh colored, rubber-like shell which is comfortable and unobtrusive. A patient also carries a small, touch-screen, remote device that communicates wirelessly with the glucose monitor and the pump system. The remote device has a form factor that can be worn or carried in various ways such as on a keychain, necklace, or armband.

In some embodiments, a method for managing diabetes proceeds as follows. A glucose monitoring system attached to a patient measures a patient's blood glucose level. The glucose monitoring system wirelessly transmits the patient's blood glucose level to a remote device. The remote device wirelessly receives the blood glucose level of the patient. Then, in some embodiments, the wireless device compares the blood glucose level to a previous blood glucose level and displays the blood glucose level trending data on its display screen.

The trending data can be displayed in the form of a trending arrow, a trending graph, a change in an ambient display, or any combination of the aforementioned options. The ambient display is customizable. The patient can choose ambient display themes such as weather, digital pet, lava lamp, or patterned colors. If a change in ambient display is used to display trending information, the display image is a positive display when the trending is in a normal blood glucose level range, and is a negative image when the trending is outside of, or nearly outside of the normal range. For example, if the ambient display is weather themed, a darker sky is displayed when trending outside of the normal range and a lighter sky is displayed when trending in the normal range. If the ambient display is digital pet themed, an unhappy digital pet is displayed when trending outside of the normal range and a happy digital pet is displayed when trending in the normal range. If the ambient display is a lava lamp theme or a colored background theme, a warm color is displayed when trending outside of the normal range and a cool color is displayed when trending in the normal range.

In some embodiments, the remote device also displays the latest blood glucose level along with the trending data. In other embodiments, the remote device displays the latest blood glucose level alone. In some embodiments, the remote device also compares the measured blood glucose level to a pre-determined dosage cycle trigger level. In some embodiments, the pre-determine dosage cycle trigger level is selected from the group consisting of: a blood glucose level, a time of day, a period of time since a previous insulin dosage, and an alert message. In some embodiments, the remote device determines whether to initiate a suggested dosage cycle based on the result of its comparison to the predetermined dosage cycle trigger level. In other embodiments, the patient separately initiates a dosage cycle. If the suggested dosage cycle is initiated, then the remote device determines a suggested insulin dosage based on the blood glucose level and a patient profile, which includes historical blood glucose levels, stored on the remote device. In some embodiments, the suggested insulin dosage suggesting is at least partially based on a patient specified mode such as a sleep mode, a rest mode, an exercise mode, a work mode, a school mode, an eating mode, and a default mode. In some embodiments, the patient then accepts, rejects, or modifies the suggested insulin dosage. In some embodiments, the patient can modify the suggested insulin dosage by inputting units of carbohydrates he anticipates eating in the near future.

The remote device then calculates an insulin dosage to be administered. In some embodiments, the insulin dosage to be administered is the same as the suggested insulin dosage; in other embodiments, they are different. In most embodiments, the patient then verifies the insulin dosage to be administered. In some basal dosages, a low level insulin drip can be automatically administered without patient verification. The remote device wirelessly transmits the dosage to be administered to a pump system attached to a patient. The pump wirelessly receives the insulin dosage to be administered and then administers the corresponding amount of insulin to the patient.

In some embodiments, the blood glucose monitoring system, the pump system, and the remote device are distinct from one another. In other embodiments, the blood glucose monitoring system and the pump system can be covered under one soft shell cover. In this embodiment the pump system and blood glucose system is called a single pump-monitor system regardless of whether or not they are embodied in a single housing.

In some embodiments, if a blood glucose level reaches below a predetermined blood glucose alert level an alert is triggered. The alert can be visual, haptic, audio, or a combination of more than one of these types. In some embodiments, similar alerts will be activated if a technical problem occurs. For example, if the insulin level in the reservoir is low, there is a malfunction in the blood glucose monitoring system, there is a malfunction in the pump system, the pump is disconnected from a cannula, a cannula is disconnected from a needle, the remote device is out of range with the blood glucose monitoring system, or the remote device is out of range with the pump system an alert will also be activated.

In some embodiments, the remote device sends, either wirelessly or by means of a USB port, a patient profile to an external computing device for tracking long term trends, tracking trends in various modes, or assisting patients in goal setting.

In some embodiments, the remote device has a touch sensitive display screen, a wireless transmitter for wirelessly transmitting an insulin dosage to be administered to a patient, a wireless receiver for wirelessly receiving a blood glucose level of a patient, a processor, a power source, and a memory comprising: an operating system, a patient profile, and a dosage calculator for calculating the insulin dosage to be administered. In some embodiments, the memory further comprises a machine learning algorithm, a patient predictive model, and a monitoring algorithm.

In some embodiments, the remote device has a USB port. In some embodiments, the remote device also has an attachment means. In embodiments with an attachment means, the remote device can be attached to a necklace, arm band, keychain, or other object. In some embodiments, the remote device's display screen rotates depending on the orientation of the remote device. In some embodiments, the remote device has a hand-held form factor. In some embodiments, the remote device is no larger than 1 in by 3 inches by ½ an inch.

In some embodiments, the pump system has a pump for injecting a patient with insulin, an insulin reservoir fluidly connected to said pump for providing said pump with insulin, a cannula fluidly connected to said pump, a needle fluidly connected to said cannula, and a wireless receiver for receiving an insulin dosage to be administered to a patient from said remote device.

In some embodiments, the glucose monitoring system has a glucose sensor for sensing a blood glucose level of a patient and a wireless transmitter for transmitting said blood glucose level of a patient to said remote device.

In some embodiments, the pump system and said glucose monitoring system are covered by a soft flesh-colored shell attached to the patient for smooth and comfortable wear. In some embodiments, the shell is substantially waterproof. In some embodiments, the pump system, glucose monitoring system, and remote device are also substantially waterproof.

In some embodiments, the wireless transmitter and the wireless receiver of the remote device, the wireless receiver of the pump system, and the wireless transmitter of the glucose monitoring system communicate by a wireless means such as infrared technology, WiFi, cellular telephone technology, radio frequency technology, or Bluetooth technology.

### BRIEF DESCRIPTION OF THE DRAWINGS

For a better understanding of the nature and embodiments of the invention, reference should be made to the Description of Embodiments below, in conjunction with the following drawings in which like reference numerals refer to corresponding parts throughout the figures.

FIG. 1 is a schematic diagram of a diabetes management system according to some embodiments.

FIG. 2 shows different perspective views of the remote device of FIG. 1.

FIG. 3 is a block diagram of the internal elements of the remote device of FIGS. 1 and 2.

FIG. 4 is a schematic diagram of a combined pump system and glucose monitor system of FIG. 1 including a soft shell mounted on a patient, according to some embodiments.

FIGS. 5A-5C show the remote device of FIGS. 1-3 attached to a keychain, an armband, and a necklace, according to some embodiments.

FIGS. 6A-6D show several themes a patient can choose for the ambient display of the remote device of FIGS. 1-3, according to some embodiments.

FIG. 7 is a flow chart illustrating a method for operating a glucose monitoring system of FIG. 1, according to some embodiments.

FIG. 8 shows screen shots of the remote device of FIGS. 1-3 showing a method for operating the remote device, according to some embodiments.

FIG. 9 shows screen shots of the remote device of FIGS. 1-3 showing various alerts, according to some embodiments.

FIG. 10 shows screen-shots of the remote device of FIGS. 1-3 showing a method for initiating dosing, according to some embodiments.

FIGS. 11A-11C are schematic diagrams of the soft shell of FIG. 4, according to some embodiments.

### DESCRIPTION OF EMBODIMENTS

In some embodiments, a diabetes management system is provided that includes a unit containing both an insulin pump system and a glucose monitor system covered by a soft shell. Systems can be incorporated in a single housing or separate shells. The pump and glucose monitor systems are monitored and/or controlled by a remote device.

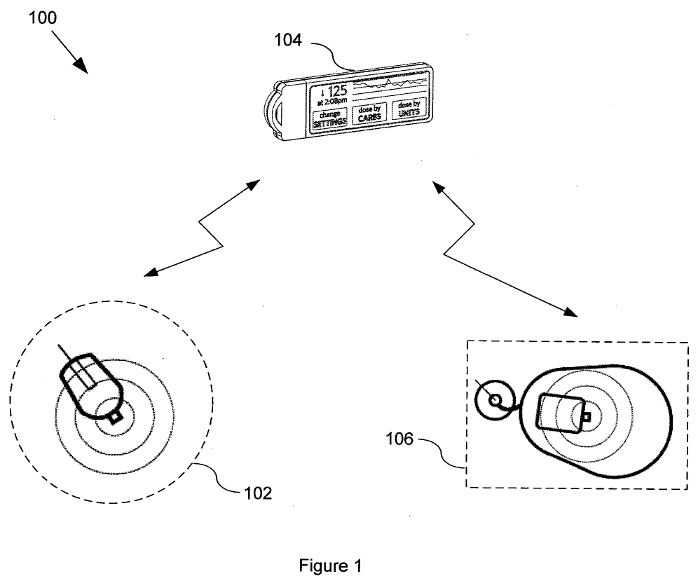

FIG. 1 is an illustration of a diabetes management system 100. The diabetes management system includes three interrelated devices, i.e., a glucose monitoring system 102, a remote device 104, and a pump system 106. The glucose monitoring system 102 periodically or continuously takes readings of the patient's blood glucose level. The glucose monitoring system 102 transmits the readings to the remote device 104. The remote device 104 receives blood glucose level readings from the glucose monitoring system 102 and stores these readings. The remote device 104 also calculates insulin dosage to be administered, either automatically or with, patient input, and transmits the insulin dosage to be administered to the pump system 106. The pump system 106 receives the insulin dosage to be administered and injects the appropriate amount of insulin into the patient. In some embodiments, the glucose monitoring system 102 and the pump system 104 can be located in the same device. In some embodiments, the communications between the pump system 106, remote device 104, and glucose monitoring system 102 occur over a wireless link. For example, infrared, Bluetooth, WiFi, radio frequency, or cellular telephone networks can be use to transmit information between the remote device 104 and the glucose monitoring system 102 and the pump system 106. In other embodiments, the information can be communicated via wires, cables, or other physical means. In some embodiments, all of the elements of the glucose monitoring system 102 are substantially waterproof.

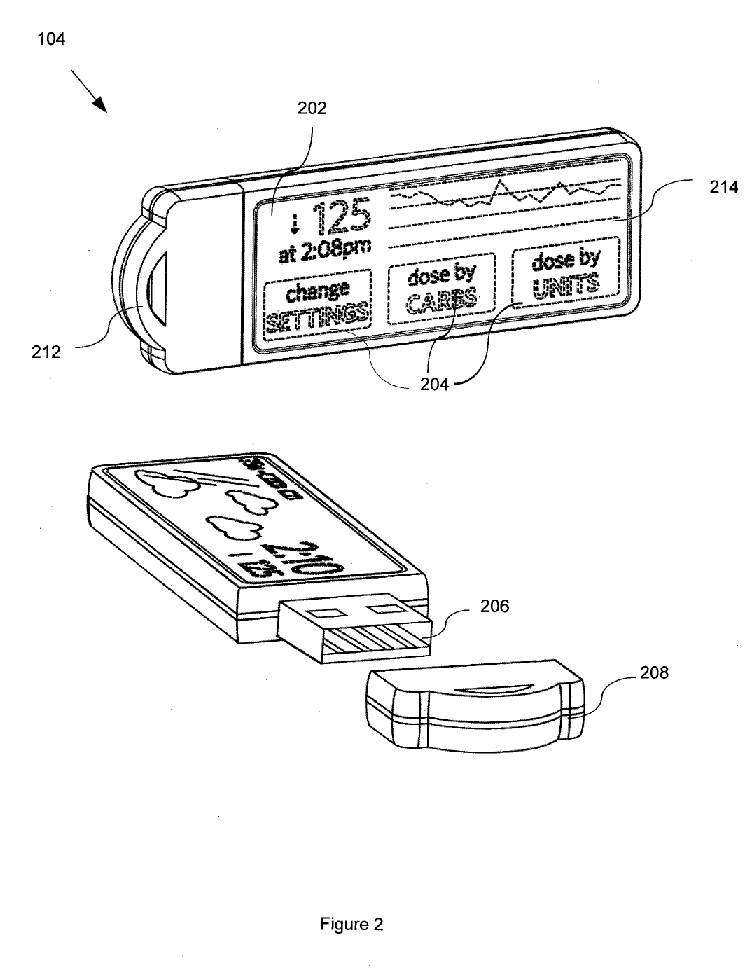

FIG. 2 shows the remote device 104 of FIG. 1. In the embodiment shown in FIG. 2, the remote device 104 wirelessly communicates with the pump system 106 and the glucose monitoring system 102. In some embodiments, the remote device has a small form factor such that it can fit easily into a patient's hand. In some embodiments, it can be at most 1 inch by 3 inches by ½ an inch. This form factor has tremendous advantages in that it is small, unobtrusive, mobile, and easy to attach to a keychain or other item already carried by the patient. However other embodiments can be larger.

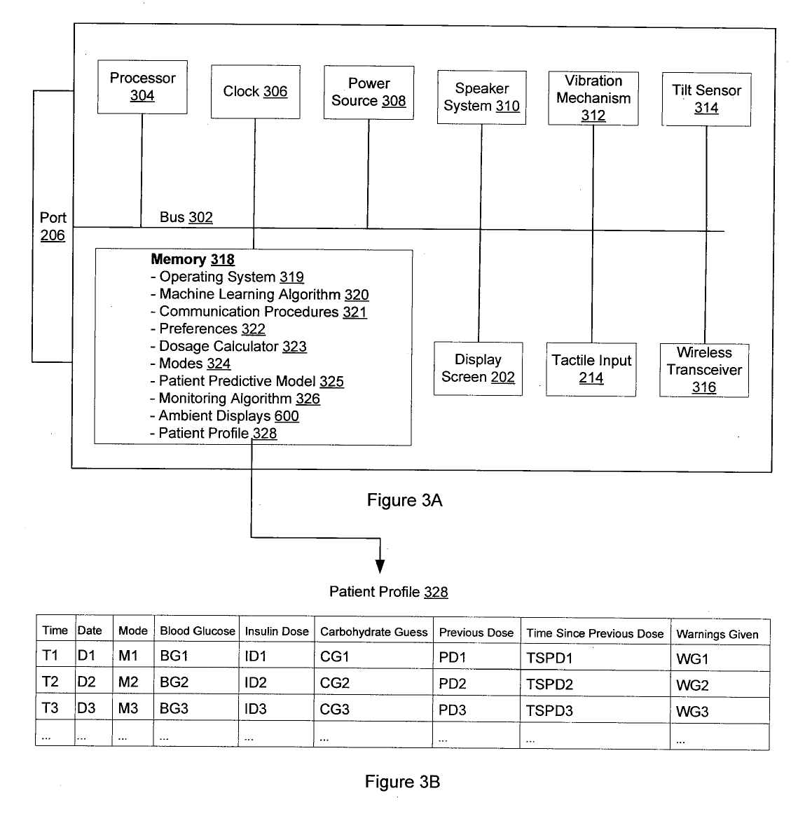

The remote device 104 embodies a display screen 202. In some embodiments, the display screen 202 is touch sensitive, i.e. it includes a tactile input 214 to allow the patient to interact with the device by tapping, scrolling, or sliding his finger on the display screen 202. For example, in the embodiment shown in FIG. 2, dynamic or soft buttons 204 appear on the display screen 202 allowing the patient to “change SETTINGS,” “dose by CARBS,” and “dose by UNITS.” In some embodiments, there is a headphone jack such that the patient can be alerted to warnings while sleeping without waking a partner. Furthermore, a patient can listen to calming or motivational music while dosing on performing other activities. In some embodiments, there are also permanent or hard buttons on the remote device 104. For example, there can be a permanent on/off button or switch, a re-start or re-boot button, a return to default settings recessed button, or any other button that might be useful to a patient without needing to interact with the tactile input 214 on the display screen 202. In some embodiments, the screen has a lock option, such that when the patient is not actively interacting with the remote device 104, the patient will not inadvertently change a setting by accidentally touching the display screen 202. The tactile input 214 can be unlocked by either a physical unlock button located on the remote device 104, or it can be unlocked by a particular stroke on the display screen 202 such as a finger slide, a tapping pattern, or a swirl motion. The unlocking method can be determined by the user and stored in the patient's preferences 322 (FIG. 3) on the remote device 104.

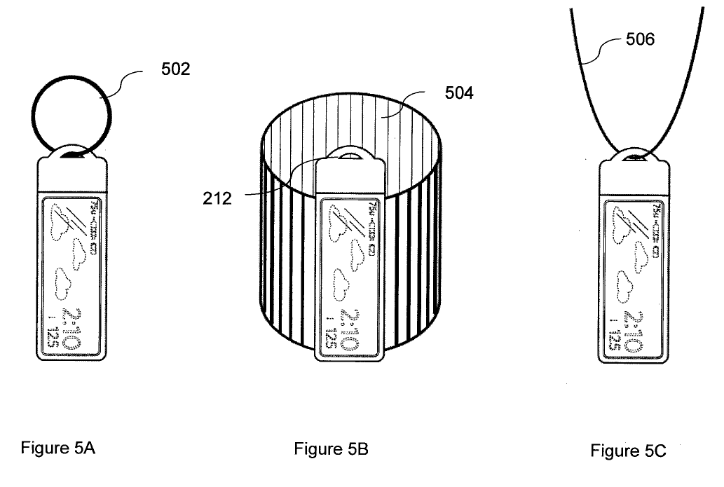

In some embodiments, the remote device 104 contains a port 206, such as a USB port, for communicating with an external computing device, such as a desktop computer. The information contained in the remote device's 104 memory can be uploaded via the port 206. Data, additional programs, features, and firmware, can be downloaded from the external computing device to the remote device 104 via the port 206. In other embodiments, the information can be communicated wirelessly and not via the port 206. In some embodiments, the port 104 is covered by a protective cap 208 that can be removed from covering the port 206. In other embodiments, the cap 208 is attached to the remote device 104 via a cord, swivel device, or any suitable means to keep the cap from becoming permanently separated from the port 206, while still allowing the port to plug into an external computing device. In some embodiments, the cap 208 can contain a loop 212 such that a necklace, cord, keychain or other mechanism can be strung through the loop 212 as shown in FIGS. 5A-5C.

Downloading the information from the remote device 104 to an external computing device, allows the patient to visualize long term trends such as month long glucose level trends or the patient's reactions to various levels of insulin doses during various modes of operation. Larger and more complex graphs and trend tracking may be available on the external computing device. The patient can compare his results with other patient's results on a virtual community of patients using the diabetic monitoring system, share best practice tips, share diabetic recipes, challenge other patient's to specific goals, etc. The patient can also send his information to a health care professional and get individualized advice without a need to visit a doctor's office or clinic. Interaction with a larger community may also help a patient remain motivated, get advice, feel supported by others in a similar situation, and set goals. Even when not communicating with a virtual community or viewing long term trends on an external computing device, the patient may find it easier to remain motivated, and set short and long term goals using the diabetic management system because of its ease of use and ability to unobtrusively integrate with the patient's lifestyle.

FIG. 3A is a block diagram of the internal elements of the remote device of FIGS. 1 and 2. The remote device 104 includes: the port 206, a processor 304, a clock 306, a power source 308, an optional speaker system 310, an optional vibration mechanism 312, an optional tilt-sensor 314, a wireless transceiver 316, a memory 318, display screen 202, and a tactile input mechanism 214, all coupled to one another via at least one bus 302. The central processing unit, or processor 304, interacts with the aforementioned components via the bus 302. The clock 306 displays the time on the display screen 202, and is required by the processor 304. In some embodiments, the clock 306 can form a part of the processor 304. The remote device is powered by a power source 308, such as a battery. If a rechargeable battery is used, it can be re-charged though the port 206.

In some embodiments, the remote device 104 contains a speaker system 310 so that audio alerts or messages can be communicated the patient. Audio alerts can be any number of tones, rings, or tunes which are customizable by the patient. The volume of the audio alert can be variable. Furthermore, additional audio alerts can be added to the memory 318 of the remote device 104. In some embodiments songs can be downloaded such that the patient can play relaxing music while administering insulin or motivating music while working out. In some embodiments, the remote device 104 contains a vibration mechanism 312 such that it can communicate haptic alerts to the patient. The haptic alerts can be of various types and intensities of vibration, can be patient customizable and/or additional haptic alerts can be downloaded into the remote device 104.

In some embodiments, the remote device 104 contains a tilt-sensor 314 or other mechanism for determining the orientation of the remote device 104. The display screen 202 may re-orient its display to match the orientation of the remote device 104. In some embodiments, the remote device 104 can also contain a digital camera, media player, etc.

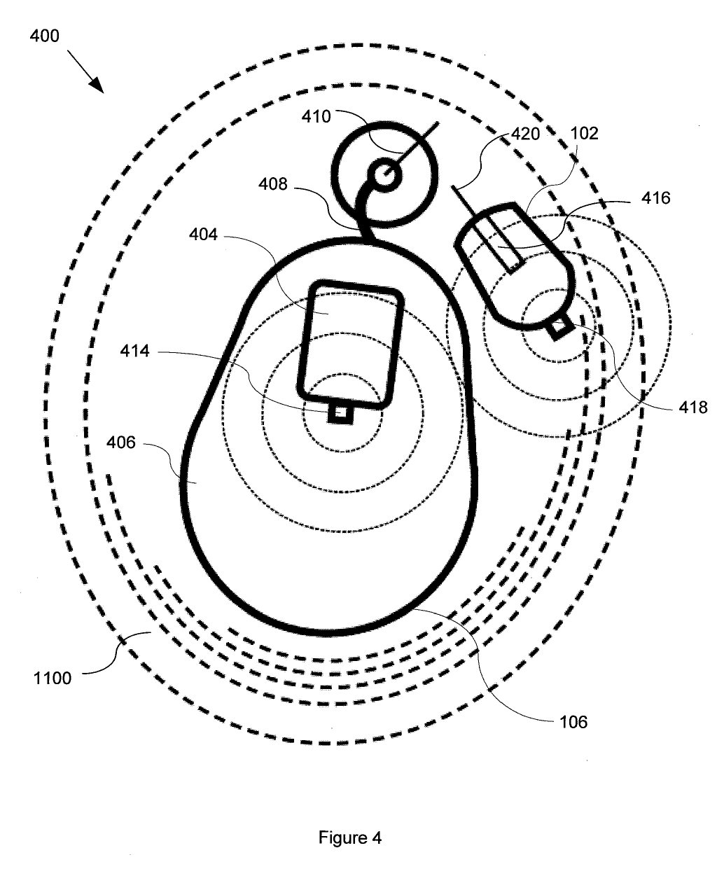

The remote device 104 contains a wireless transceiver 316. This wireless transceiver 316 automatically communicates with the transceiver 414 of the pump system 106 and/or the wireless transceiver 418 (FIG. 4) of the glucose monitor system 102. In some embodiments, the pump system 106 may contain only a receiver for receiving instructions from the remote device 104. Likewise, in some embodiments, the glucose monitor system 102 may contain only a transmitter for sending information to the remote device 104. As such, if a communication is not successfully transmitted or received, the remote device 104 will be alerted. If no transmission is possible, there is likely a malfunction; the remote is out of range with the combined pump monitor 400; or another error has occurred. The remote device 104 will activate an alert by a combination of one or more of visual, haptic, or audio alerts to notify the patient of the condition. The remote device is also capable of receiving a manual input of a blood glucose level reading. This option is useful if a manual glucose reading is taken to double check the glucose monitor system's readings. Manual input is also useful for calibration of the diabetes management system 100.

The memory 318 contains a number of elements. In some embodiments, the memory 318 contains one or more of the following: an operating system 319 that stores instructions for communicating, processing data, accessing data, storing data, searching data, etc.; a machine learning algorithm 320; communication procedures 321; preferences 322; a dosage calculator 323; modes 324; a patient predictive model 325; a monitoring algorithm 326; ambient displays 600; and a patient profile 328. The communication procedures 321 facilitate communication with the glucose monitor system 102 and the pump system 106. The dosage calculator 323 calculates patient inputted carbohydrates and produces suggested insulin dosages.

The memory 318 also contains a patient profile 328 shown in FIG. 3B. The patient profile 328 contains all of the raw data collected on a patient. The patient profile 328 stores historical information received from the glucose monitor system 102. The patient profile 328 may contain the time, date, and mode of operation each time a blood glucose level is received from the blood glucose monitoring system 102. Furthermore, the patient profile 328 contains the time, date, and mode of operation every time an insulin dosage is transmitted to the pump system 106. The patient profile 328 contains data for both monitoring algorithm 326 initiated and patient initiated doses for both basal and bolus insulin doses. Furthermore, in some embodiments, for each event, the time since the previous dose, the previous carbohydrate consumption guess, the amount of the previous dose, and any warnings or alerts that were given are also recorded and stored in the patent profile 328.

The information stored in the patient profile 328 is used to create a patient specific predictive model 325. The machine learning algorithm 320 builds the predictive model 325. The patient predictive model 325 is generated from the historical data stored in the patient profile 328 to model how a patient reacts to a certain dosage, at a certain time, and mode. For example, it may fit a curve to the raw data, or it may use any other suitable statistical or neural technique to generate the patient predictive model 325.

The monitoring algorithm 326 receives blood glucose readings and checks these readings against pre-determined dosage cycle trigger levels for the current mode 324 stored in the memory 318. The monitoring algorithm 326 initiates a dosing cycle if a predetermined dosage cycle trigger level is reached. In some embodiments, the monitoring algorithm 326 is triggered by a low blood glucose level. In other embodiments, the monitoring algorithm 326 is triggered by a particular time of day. For example, in some embodiments, the monitoring algorithm 326 will initiate a dosing cycle at noon because the patient usually eats at that time.

Once the dosing cycle is initiated, the dosage calculator 323 uses the patient predictive model 325 to generate a suggested insulin dosage. The dosage calculator 323 accomplishes this by using the patient predictive model 325 to determine the typical dosage that has been administered in the past for a blood glucose level at a given time and mode.

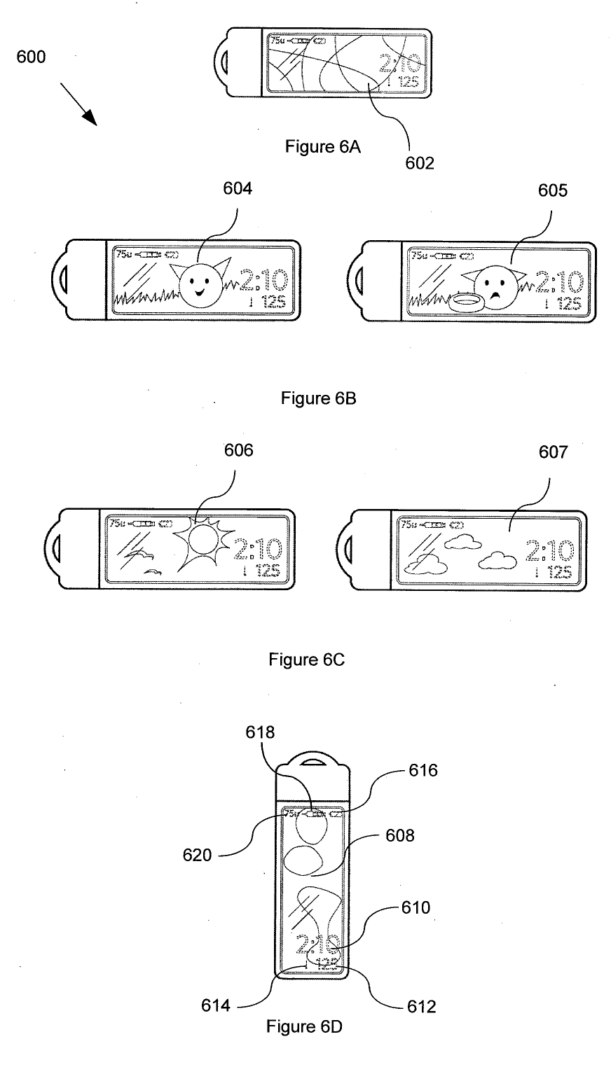

The memory 318 may contain preferences 322 such as the patient's preferred audio alerts, visual alerts, haptic alerts, ambient displays, the text font and size, basal dosage, glucose monitoring schedule, and any number of other elements that the patient has optionally selected and stored. Many of these preferences can be linked to particular modes 324. Modes 324 are the pre-set options regarding glucose monitoring, glucose displaying, insulin dosing, alerts levels, the alert type, alert volume or intensity, ambient displays, and other options that are pre-set for a standard repeatable situation. Setting-up modes 324 saves the patient time customizing particular preferences. A patient can utilize as many or as few modes 324 as desired. Some modes 324 are pre-set but customizable, such as sleep mode, exercise mode, school mode, work mode, neutral mode, default mode, emergency mode, etc. The patent can also create new modes 324. The modes can be pre-set for a 24 hour period, a weekly period, a monthly period, or a combination thereof. For example, a patient may wish to pre-assign a particular mode during the times the patient is normally sleeping, eating, and involved in work or school activities. Alternatively, a patient may wish to program a slightly different mode pattern on the weekends when he stays up later and goes to a weekly exercise class. Ambient displays 600 are also stored in the memory 318. Some examples of ambient displays 600 are a color pattern, a digital pet, weather, and a lava lamp as discussed in relation to FIGS. 6A-D.

The remote device 104 may also contain additional software programs. For example, it may contain a calendar, an address book, or the option to send and receive information such as e-mail wirelessly for sending immediate readings to a health care professional, for requesting advice, or for setting up future appointments. The remote may also contain a picture folder for customizable ambient displays 326, a calculator for manual insulin dosage calculation, or any other useful program to assist the patient in operating the diabetes management system 100. In some embodiments, the memory 318 may also contain a telephone means, which utilizes the speaker system 310 and tactile input 214 to call for help. In some embodiments, in the case of an emergency, such as the patient passing out, the telephone will automatically connect to a cellular telephone network and transmit an emergency message to a trusted friend, relative, or health care professional.

In some embodiments, the remote device 104 can act as not only a communication device with the glucose monitor system 102 and the pump system 106, but also as a complete personal digital assistant for a patient's daily life. This reduces the number of items that a patient needs to carry on a daily basis. Alternatively, in some embodiments, the functions of a remote device 104 may be transferred to an alternative handheld device such as a cellular telephone or personal digital assistant. In these embodiments, the alternative handheld device may perform the functions of the remote device 104 described above. The screen, speaker, clock, vibration mechanism, and memory of the alternative handheld device may be utilized. The pump system 106 and the glucose monitoring system 102 wirelessly communicate with the alternative device by infrared, Bluetooth, WiFi, or cellular telephone networks.

FIG. 4 shows an embodiment wherein a soft shell 1100, shown in dashed lines in this figure, covers a pump system 106 and glucose monitor system 102 on a patient's body. When the pump system 106, and glucose monitor system 102 are both positioned under the same shell 1100, the elements under the shell 1100 are called a combined pump-monitor 400, even when the pump system 106 and the glucose monitor system 102 are not physically combined within a single housing. In some embodiments, the combined pump-monitor 400 will include a glucose monitor system 102 physically embodied within the pump system 106. In these embodiments, the needle 410 for the pump system 106 also contains a glucose sensor 416, such that only one needle is inserted into the patient.

In some embodiments, the glucose monitor system 102 is positioned a certain distance away from the pump system 106 to facilitate obtaining a more accurate blood glucose level reading. Therefore, in some embodiments, the glucose monitor system 102 is positioned on another part of the patient's body, and not under the same shell 1100 as the pump system 106. In those embodiments, the glucose monitor system 102 can be positioned under a separate soft shell 1100 or may not be covered by a shell at all.

The glucose monitor system 102 includes a glucose sensor 416, a needle 420 and a transmitter or transceiver 418, as shown in FIG. 4. During a typical glucose monitoring operation the glucose sensor 416 measures the patient's blood glucose level using a small portion of blood obtained by the needle 420. The transmitter or 418 wirelessly transmits the blood glucose level to the remote device 104 (FIG. 1). The glucose monitoring system 102 can be worn continuously over a period of time without having to be repositioned. In some embodiments, that period of time is one day, one week, one month, or even longer.

The pump system 106 includes a pump 404, an insulin reservoir 406, one or more cannula 408, one or more needles 410, and a transmitter or transceiver 414. Unlike current products, in some embodiments, the reservoir 406 is soft such that it appears less bulky under the shell 1100. In other embodiments, the reservoir 406 is hard, but due to its snug fit inside the shell 1100, the reservoir 406 appears soft. In some embodiments, the reservoir 406 is refilled by injection through the shell 1100. In some embodiments, the pump 404 and/or insulin reservoir 406 can be permanently or semi-permanently embedded in the shell 1100. In some embodiments, the pump 404 can easily slide off of the cannula 408. The pump system 106 can be worn continuously for a period of time without having to be repositioned. In some embodiments, that period of time can be one day, one week, one month, or even longer.

During a typical dosing operation, the pump's transceiver 414 receives an actual dosage transmission from the remote device 104 (FIG. 1). The transceiver 414 communicates the appropriate dosage to the pump 404. The pump 404 then transfers the appropriate amount of insulin from the insulin reservoir 406, through the cannula 408, and into the needle 410 such that the appropriate amount of insulin is administered to the patient. In some embodiments, after the dosage has been administered to the patient, the transceiver 414 transmits the amount of insulin that was delivered back to the wireless device 104. In some embodiments, if the insulin reservoir 406 is out of insulin, if the needle 410 has disengaged from the patient, if the cannula 408 has detached from the needle 410 or the pump 404, or if any other malfunction occurs that could affect the insulin dosage to the patient, the transceiver 414 will communicate an appropriate warning or alert to the remote device 104.

The shell 1100 allows the pump system's transceiver 414 and the glucose monitor system's 102 transceiver 418 to wirelessly communicate with the remote controller 104 (FIG. 1). As such, during normal operation the patient does need to manually interact with either the glucose monitor system 102 or the pump system 106. This makes glucose monitoring and insulin dosing easy for a patient to check and administer discretely from almost anywhere.

FIGS. 5A-5C show the remote device 104 attached to a keychain 502, an armband 504, and a necklace 506 respectively. In other embodiments, the remote device 104 is attached to a shoe lace, a cord or ribbon on a coat, a backpack, purse or any other means to keep the remote device 104 near a patient. The remote device 104 can also be carried loose in a pocket, purse, or by other means. The small form factor and durability of the remote device 104 allows it to be easily carried with the patient. It can attach to anything small enough to fit through the loop 212 on the cap 208, as for example, the keychain 504 and the necklace 506 fit through the loop 212 in FIGS. 5A and 5C respectively. In some embodiments, the loop 212 is disposed on the body of the remote device 104, rather than on the cap 208.

The keychain 502 of FIG. 5A can be made of any suitable material for sliding through the loop 212. For example, it can be made of metal, plastic, fabric, or any similar material. The keychain 502 can be of any shape and is not required to be round. The keychain 502 embodiment allows the remote device 104 to be attached to other items, and may not necessarily also hold keys. For example, a carabineer might be used to attach the remote device 104 to a patient's belt or clothing.

The armband 504 of FIG. 5B can be made of any suitable material such as fabric, plastic, elasticized material, or metal. In FIG. 5B, the armband 504 is attached to the back of the remote device 104. In some embodiments, this attachment is accomplished by Velcro, snaps, loops, or any similar attachment means for securing the remote device 104 to the body of the armband 504. In other embodiments, the remote device 104 can slip into a pocket in the armband 504. The armband 504 need not only be attached to a patient's arm, but can also be worn on the wrist, around the ankle, upper arm, or around other part of the patient's body. Furthermore, the armband 504 can be used to attach the remote device to another object such as a backpack strap, purse strap, can be used as a coffee mug sleeve, or any suitable item which the armband 504 can wrap around. In some embodiments, the armband 504 contains a means for attaching and detaching from itself such that it can wrap around the patient's arm or another object. For example, it can have snaps, Velcro, or a buckle attachment means.

The necklace 506 of FIG. 5C can be made of any suitable material such as a plastic cord, a metal chain, a ribbon, or fabric cord. The necklace 506 can be worn around the patient's neck, worn around the patient's waist, wound around the patient's wrist, tied in a patient's hair, or worn in any other suitable method. The necklace 506 can also be tied or draped over another object such as tied onto a handlebar of a bicycle, backpack, baby stroller, or any suitable object. The necklace 506 cord can be attached through the remote device's loop 212, or can be attached to the remote device 104 by another means. For example, it can attach at the back of the remote device, such that the device hangs in a landscape orientation when worn around the neck of a patient. In some embodiments, it slides into a pocket in a thick necklace 506 cord.

These attachment means are provided as examples only. Any other attachment devices that provided a mechanism for a patient to carry the remote device can also be used. For example, in other embodiments, the functionality of the remote device may be embodied inside a watch, such that the patient may wear it on his wrist, and the ambient display is a watch face when not actively communicating diabetes related information to the patient.

FIGS. 6A-6D show various sample ambient display background image themes. The sample themes include an abstract design themed ambient display 602 in FIG. 6A, a digital pet themed ambient display 604 in FIG. 6B, a weather themed ambient display 606 in FIG. 6C, and a lava lamp themed ambient display 608 in FIG. 6D. In other embodiments, themes include an aquarium theme, flower theme, beach theme, a nature theme, and a solid color or pattern. A person skilled in the art would recognize than many other themes are also possible. Furthermore, a patient can download other ambient display 600 themes not-pre-loaded onto the remote device 104. A patient can also customize the ambient display with a personal photograph or other background.

As shown in FIGS. 6B and 6C, in some embodiments the ambient displays changes to subtly show a patient blood glucose level trending information. For example, when a patient's blood glucose level is trending downward; the ambient display 600 changes its image to subtly alert the patient that negative trending of blood glucose levels is occurring. For example, in some embodiments, the digital pet looks happy 604 when the blood glucose levels are in the normal range. The digital pet looks sad 605 and shows an empty food dish if the blood glucose levels are outside of the normal range. Another example, show in FIG. 6C, is for a weather themed ambient display 606. Normal blood glucose levels are shown by a sunny sky 606, and abnormal blood glucose levels can be shown as a cloudy sky 607. In some embodiments, color changes are used to indicate changes in blood glucose levels. For example, warm colors like yellows and reds are used to indicate abnormal blood glucose levels, and cool colors like blues and purples are used to indicate normal blood glucose levels in the abstract design theme 602 of FIG. 6A and the lava lamp theme 608 of FIG. 6D. This method for alerting a patient to negative or positive trending is especially useful for patients, such as children, who cannot read. In some embodiments, the change in ambient display can be set to show a negative trending situation, even if the blood glucose range is still normal, thus the ambient display gives a patient advanced warning of a potential need to adjust his blood glucose level before adjustment is immediately necessary.

In some embodiments, the orientation of the ambient display is landscape view as is shown in FIGS. 6A-6C. In some embodiments, the ambient display is portrait view, as is shown for the lava lamp themed ambient display 608 in FIG. 6D. In some embodiments, the background image can orient itself in either landscape or portrait depending on the orientation of the remote device 104 at the time of viewing.

In some embodiments, the ambient display includes icons conveying general information along with a background image. For example, FIG. 6A shows the time 610, blood glucose level 612, blood glucose trending information 614, battery life 616, visual indication of approximate amount of insulin left in the insulin reservoir 618, and the numerical amount of insulin left in the insulin reservoir 620. This additional information provides the patient with a variety of information at a glance. The patient can customize the ambient display to show as many or as few of these icons as the patient prefers. The patient can also customize the size of these icons. For example, if the patient finds it hard to read the print size of the amount of insulin left in the insulin reservoir 620, he or she can choose to customize the print size to be larger. Other icons can also be displayed in the ambient display. For example, the patient may wish to display an icon every time an automatic basal insulin injection has occurred. A patient may also wish to display a timer showing how long it has been since the last bolus or basal injection has occurred.

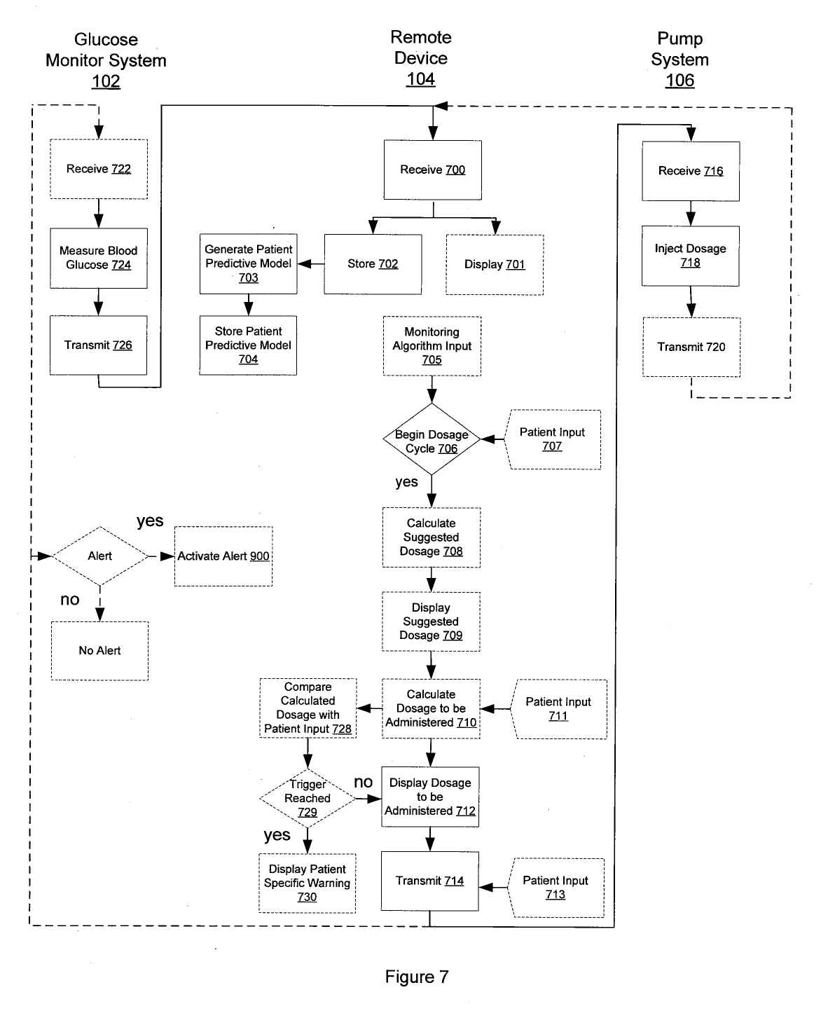

FIG. 7 schematically illustrates a typical monitoring and dosing cycle. The monitoring cycle proceeds as follows. The glucose monitoring system 102 measures a blood glucose level at 724. This may be because of an instruction to measure received from the patient at 722. Alternatively, measuring the blood glucose level at 724 may be performed periodically and automatically. Blood glucose level measurements can be taken every minute, every five minutes, every ten minutes, once an hour, several times a day, once a day, or the like. The glucose monitoring system 102 then wirelessly transmits the blood glucose level to the remote device 104 at 726. The blood glucose level can be transmitted continuously or periodically. For example, blood glucose level transmissions can occur every minute, every five minutes, every ten minutes, once an hour, several times a day, once a day or the like.

The remote device 104 receives a reading of the current glucose level from a glucose monitoring system 102 at 700. The remote device 104 stores at 702 this glucose level in its memory 318 (FIG. 3A), along with the date, time, and mode settings at the time of the glucose reading as a part of a continuously updated patient profile 328 (FIG. 3B). The remote device generates at 703 on updated patient predictive model 325 (FIG. 3A) based on the newly stored information and the information in the patient profile 328. Then the updated patient predictive model 325 is stored at 704.

In some embodiments, at any time after receiving the blood glucose level at 700, the remote device 104 displays the current blood glucose level reading on a display screen at 701, either as an individual glucose level or an average glucose level over a period of time. An average glucose level display protects against statistical variations resulting from an individual glucose level measurement malfunction. The blood glucose level can be displayed continuously or periodically. For example, the blood glucose level can be displayed every minute, every five minutes, every ten minutes, once an hour, several times a day, once a day, or the like. The patient can specify how often the readings are taken and also how often the readings are displayed. In some embodiments, trending information of the current glucose measurement as compared to the previously displayed glucose measurement is displayed in the form of a graph, arrow, or change in ambient display 600. In some embodiments, the trending information can be displayed only as a change in ambient display 600 (FIG. 6). In other embodiments, the trending information can be displayed simultaneously with the glucose reading or average glucose reading.

The dosage cycles described below normally apply to bolus dosages, although they can apply to basal dosages as well. A basal dosage is an insulin dosage lower than a bolus dosage. A bolus dosage is usually used to cover high insulin needs such as during mealtimes. A basal dosage is often set up to automatically deliver insulin between meals or at times of rest. It is not necessary that both a bolus and a basal dosage be given to a particular patient. For example, one patient may only require basal level doses while another patient may need only bolus doses. However, some patients require both basal and bolus doses.

In some embodiments, patient input at 707 begins a dosage cycle at 706. In other embodiments, the monitoring algorithm 326 (FIG. 3A) at 705 initiates a dosing cycle at 706 based on a trigger. Periodically, the patient profile 328 is read by the monitoring algorithm 326. For example, the patient profile 328 can be read every minute, every five minutes, every ten minutes, once an hour, several times a day, once a day, or the like. The monitoring algorithm 326 monitors the patient's blood glucose level, time, and other changing situation to see if they fall below or above a dosage cycle trigger level. In some embodiments, if a patient's blood glucose reading falls below a low blood glucose trigger level, then the monitoring algorithm 326 initiates a dosing cycle at 706. In some embodiments, the monitoring algorithm 326 initiates a dosage cycle at 706 based on another trigger such as by a particular time of day; time since previous insulin dosage; an alert message; or the like. Setting the dosage cycle trigger level assists the patient in goal setting. For example, if the patient wishes to more closely control his or her blood glucose level, he could set a trigger to always begin dosing cycle if I reach 120 mg/dL.

In the embodiment where the decision to begin a dosage cycle at 706 is initiated by the monitoring algorithm 326, the remote device 104 then calculates, at 708, a suggested insulin dosage. The suggested insulin dosage is calculated at 708 based on the received blood glucose level 700 and the patient predictive model 325 (FIG. 3A), stored at 704, using the dosage calculator 323 (FIG. 3A). The remote device 104 then displays the suggested insulin dosage at 709. In some embodiments, input is received from the patient to accept, reject, or modify the suggested insulin dosage at 711. If the patient chooses to modify the dosage, the patient can modify it by either inputting units of insulin or inputting units of projected carbohydrates that the patient plans to eat. The remote device 104 then uses the dosage calculator 323 to calculate at 701 the insulin dosage to be administered at 710. The remote device 104 then displays an insulin dosage to be administered at 712.

In some embodiments, the calculation of the insulin dosage to be administered at 710 is determined by the patient input approving or modifying the suggested insulin dosage at 711. In other embodiments, if no patient input 711 is received after a pre-determined amount of time has passed the remote device 104 determines that the insulin dosage to be administered 710 is calculated as the suggested insulin dosage 708. In yet other embodiments, the patient can select the mode 324 (FIG. 3A) such that the insulin dosage to be administered 710 is always the suggested insulin dosage at 710 such that the step of displaying the suggested insulin dosage at 709 is skipped. In this embodiment, only the insulin dosage is displayed 712.

In the embodiment where the decision to begin a dosage cycle at 706 is initiated by the patient at 707, the following steps vary depending on the original patient input at 707. In some embodiments, the patient initiates the dosage cycle at 706 by simply entering instructions to begin a suggested insulin dosage calculation at 707. In this embodiment, the steps are the same as described above for the dosage cycle initiated by the monitoring algorithm 326.

In some embodiments, the patient enters the approximate amount of carbohydrates that the patient plans to eat at 707. The remote device 104 calculates the suggested insulin dosage, at 708, using the dosage calculator 323 (FIG. 3A) and taking into account not only the patient inputted carbohydrate amount but also the received blood glucose level 700 and the patient predictive model 325 (FIG. 3A), stored at 704. The suggested insulin dosage is displayed at 709. The patient then accepts, rejects or modifies the suggested dosage at 711. The dosage calculator 323 then determines the insulin dosage, to be administered at 710, and displays it at 712.

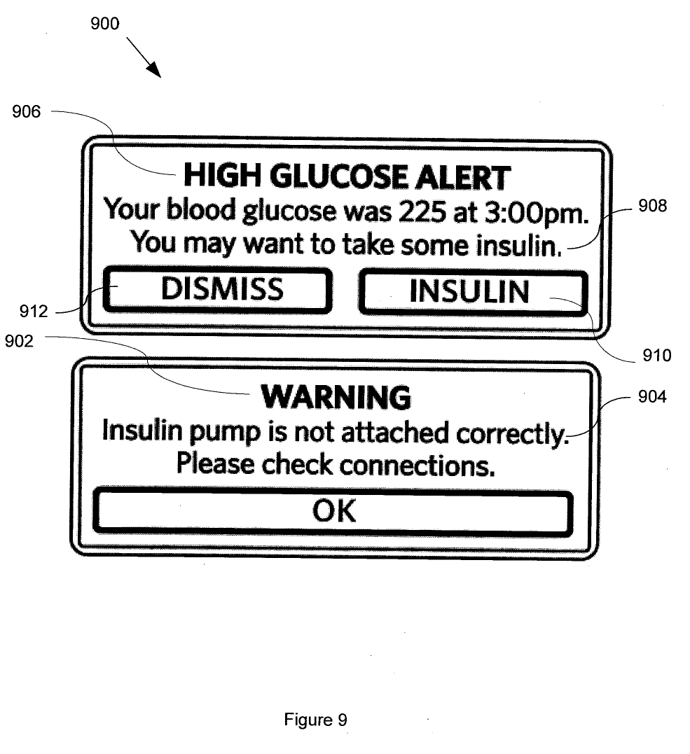

In other embodiments, the patient enters the amount of insulin units the patient would like to receive at 707. In this case no suggested dosage need be calculated at 708 or displayed at 709. In some embodiments, the remote device 104 proceeds immediately to the step of displaying the insulin dosage to be administered at 712. In other embodiments, the remote device calculates at 710 the insulin dosage to be administered based on the received blood glucose level 700 and the patient predictive model 325 (FIG. 3A), stored at 704. The calculated value, determined at 710, and the original patient inputted value, received at 707, are compared at 728. The comparison value is checked against a warning trigger level at 729. If a warning trigger level is not reached, i.e. if the calculated value for the insulin dosage to be administered 710 falls within a preset range of values close to what the patient inputted at 707, then the amount that the patient initially entered is displayed at 712. However, if the warning trigger level is reached, i.e. if the value calculated at 710 differs substantially from the value inputted by the patient at 707, then a patient specific warning 906 (FIG. 9) informing the patient that the amount of insulin that the patient entered is potentially incorrect is displayed at 730. This double check mechanism helps protect a patient from accidental insulin overdosing. In some embodiments, a similar patient specific warning 906 appears if the approximate amount of carbohydrates is substantially different from the patient's normal intake of carbohydrates. In some embodiments, the patient specific warning 906 trigger levels are pre-set by a medical professional. In other embodiments, the patient can set the trigger levels for when these patient specific warnings 906 are displayed.

No matter how dosage cycle is initialed, for the safety of the patient, prior to transmitting the actual insulin dosage for a bolus level injection, the patient confirms that the dosage should be administered at 713. This is a safety feature to protect the patient from a potentially lethal overdose of insulin. However, certain low level basal injections can be set to administer by drip. In some embodiments, basal injections by drip do not require patient input at 713 before transmission. This can be especially helpful if the patient is sleeping or otherwise engaged and does not wish to be bothered for low level basal injections. Finally, the actual dosage is wirelessly transmitted, at 714, to the pump.

The pump system 106 (FIG. 1) receives, at 716, from the remote device 104 the insulin dosage to be administered. The pump system 106 then injects the appropriate amount of insulin into the patient at 718. In some embodiments, the pump system 106 then transmits at 720 injection information back to the remote device 104. For example, if the injection is successful, the pump system 106 transmits a success message, or if an injection malfunction occurred, an alert is transmitted 720.

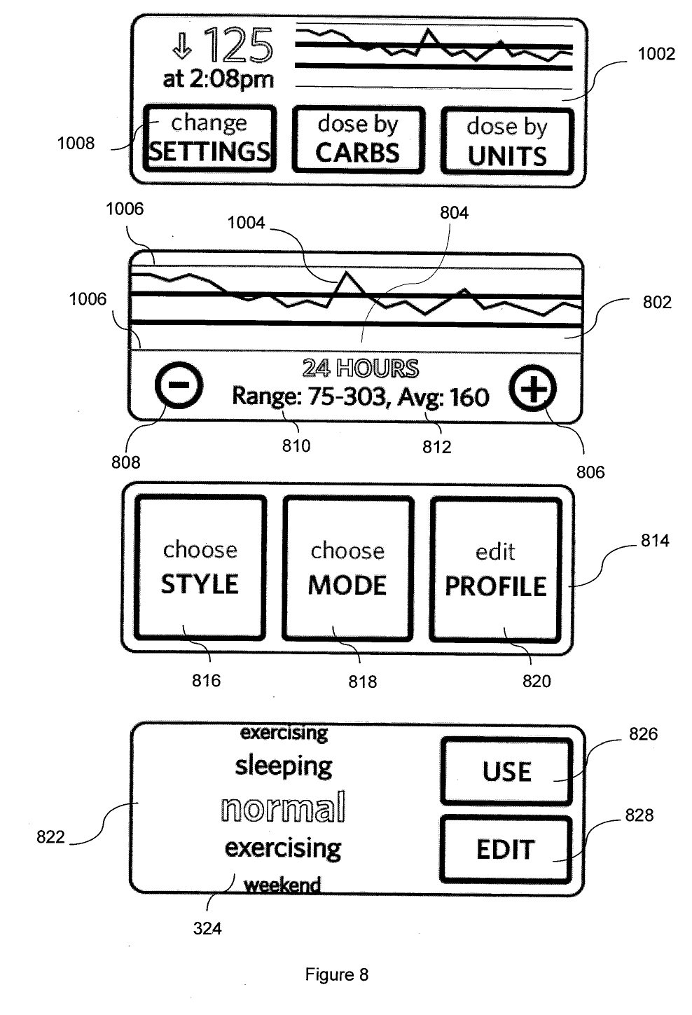

FIG. 8 shows various interface screens that may appear while a patient is using the remote device 104. Following unlocking, the landing screen 1002 appears. The patient can tap on the graph 1004 to proceed to the glucose overview screen 802. At the glucose overview screen 802, the patient can view and change the time period 804 of the graph 1004. In some embodiments, the time period of the graph 1004 is changed using the plus key 806 and the minus key 808. The upper and lower limit guide lines 1006 are modified by similar means. Other information regarding the graph may also be displayed. For example, in the embodiment shown in FIG. 8, the blood glucose range 810 and the blood glucose average 812 are displayed. Similar information as specified by the patient such as the percentage of time that the patient remained within the specified upper and lower guide lines 1006 can also be shown.

If the patient chooses the “change SETTINGS” 1008 soft button 204 (FIG. 2) in the landing screen 1002, the patient will be taken to the menu screen 814. In the menu screen 814, several options are available to the patient such as the soft buttons 204 for “Change STYLE” 816, “Change MODE” 818, and “Change PROFILE” 820. If the patient chooses the “Change STYLE” button 816 the patient is taken to a Style screen, not shown, where in some embodiments, the patient can change the ambient display, change the font, color, or size of the display text, change the shape of the options buttons, or any similar settings which relate to the visual style of the display screens. The patient can adjust these levels by various tactile input 214 methods such as tapping, scrolling, or sliding their finger up and down on the touch sensitive display screen 202.

If the patient chooses the “change MODE” button 818, the patient is taken to the Mode screen 822 where, in some embodiments, the patient can chose modes 324 (FIG. 3) of operation by taping on the mode menu options 324. Different modes 324 can be stored, such as sleep, exercise and normal. Once the proper mode 324 is selected, the patient will choose the “USE” button 826 to start using this mode 324. For example, the patient may change to the sleep mode 324 right before bed.

The patient can also edit the modes by selecting the “EDIT” button 828. In some embodiments, the patient can pre-set a variety of options for each mode 324, such as the dosage for a basal injection, how often the basal injection should be administered, and how often to check the patient's blood glucose level. In some embodiments, the patient can pre-set a schedule for daily mode 324 changes. For example, the patient can set a 24 hour period of time with a sleep mode 324 from 11 PM to 6 AM, an eating mode 324 from 6 AM to 7 AM, a work mode 324 from 7 AM until NOON, a eating mode 324 from NOON to 1 PM, work mode 324 from 1 PM to 6 PM, an exercise mode 324 from 6 PM to 7 PM, eating mode 324 from 7 PM to 8 PM, and a leisure mode 324 from 8 PM to 11 PM. In this embodiment, the patient will not need to manually change modes 324 before eating, going to work, bed, etc.

FIG. 9 shows two examples of visual alerts 900 that can appear in special circumstances. In some embodiments, the visual alerts 900 are also be accompanied by audio and/or haptic alerts. In other embodiments, an audio and/or a haptic alert is used without a visual alert 900. The visual alerts 900 background colors can be red or another eye catching color.

Some alerts warn of a device malfunction or technical problems 902. These can relate to any technical problem occurring with the diabetes management system 100 (FIG. 1). One example, shown in FIG. 9, is a pump disconnection warning 904. Other technical problems 902 including, a low insulin level in the insulin reservoir 306, a malfunction of the glucose monitor, a malfunction of the pump 304, a low battery, a needle that has detached from the patient, or any similar physical malfunction will also trigger a malfunction warning 902. Another example of a malfunction 902 that causes an alert occurs when the remote device 104 is moved out of range with the pump system 106, the monitor system 102, or the combined pump monitor 400 (FIG. 4), because although all of the physical elements are functioning properly individually, they cannot properly perform their functions unless they are able to communicate with one another.

Another type of visual alert is a patient specific warning 906. For example, the alert shown in FIG. 9 conveys information regarding a high glucose reading 908. Other patient specific warnings 906 include a low glucose reading alert, a potential overdose of insulin, an unusual trending cycle as compared to the patient profile, a warning that the amount of insulin or the number of carbohydrates that the patient entered is potentially incorrect, and any other similar patient specific issues that demand a patient's attention. For example, if a patient attempts to dose himself with a large bolus insulin dosage shortly after administering a previous large bolus insulin dosage, a high insulin dosage alert appears warning the patient that the current insulin in his body could reach too high of a level if the new bolus were administered.

The patient can choose to perform a function to correct the problem. For example in FIG. 9, the patient can choose the “INSULIN” button 910 to begin a dosage cycle 705 (FIG. 7). Alternatively, the patient can choose the “DISMISS” button 912 to dismiss the warning screen. In some embodiments, after dismissal, the patient specific warning 906 will permanently disappear, while in other embodiments, the warning 906 may temporarily disappear, and will reappear if the problem persists. In other embodiments, the warning will re-appear if the next blood glucose reading is a pre-determined amount lower than the previous blood glucose level. In still other embodiments, choosing the “DISMISS” button 912 will display a screen where the patient can choose whether to permanently dismiss or temporarily postpone the warning. Similar options are also available for all technical problem alerts 902.

In some embodiments, patient warnings 906 are customized to the patient. For example, the patient can set the high glucose alert to only appear if two or three glucose readings in a row appear outside of a pre-determined range used for patient warnings 406. In other embodiments, the patient customizes the pre-determined range. In yet other embodiments, the warnings become patient specific by means of the machine learning algorithm 320 (FIG. 3). In some embodiments, a warning appears if a certain small problem that was previously a precursor to a larger problem recurs. For example, if a patient's blood sugar trends up quickly at around noon, it may indicate that the patient forgot to inject himself with a bolus dosage prior to lunch, as happened on a previous day at around the same time. Thus, even though the blood glucose level has not yet exceeded the pre-determined range, a warning can appear that alerts a patient to a potential need to perform a bolus injection.

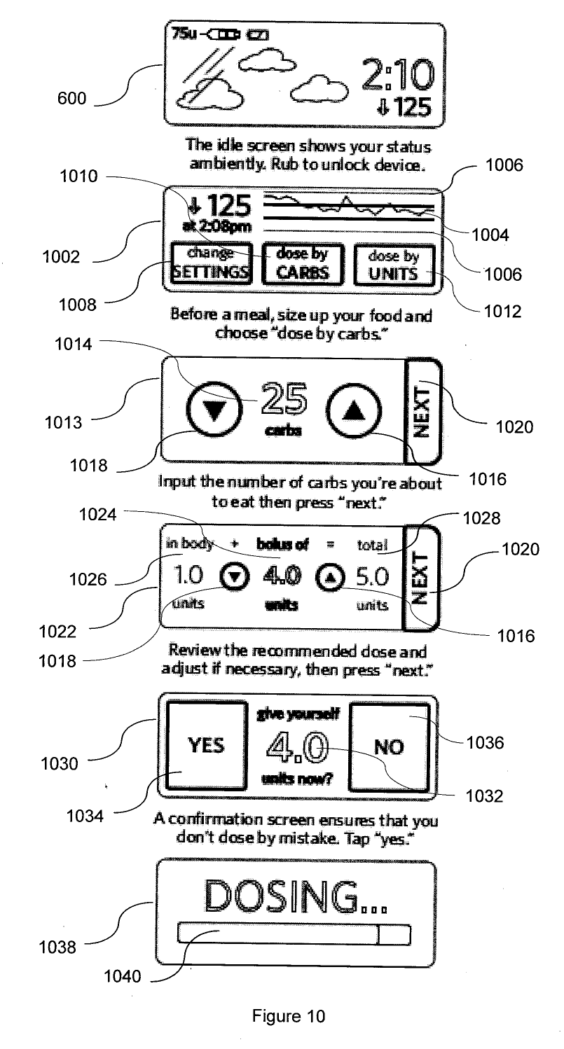

FIG. 10 shows various interface screens that may appear as a patient interacts with the remote device 104 (FIG. 1) to calculate an insulin dosage. Once a patient unlocks the ambient display 600 (FIG. 6) the landing screen 1002 will appear. In some embodiments, unlocking will occur through pressing a button or flipping a switch located on the body of the remote device 104 separate from the display screen 202 (FIG. 2). In other embodiments, unlocking will be accomplished through tapping, sliding, swirling or another operation performed on the touch screen itself. In some embodiments, the patient can customize the unlocking stroke.

In the landing screen 1002, the patient will get more information about his or her blood glucose levels over a period of time in the format of a graph 1004. In some embodiments, this graph 1004 is customized to be a part of the ambient display 600. The period of time that the graph covers can be altered by the patient, such as the last hour, the last day, or the last week. In some embodiments, the graph also shows guide lines 1006. The guide lines 1006 mark the range of blood glucose levels that the patient wishes to stay between. In some embodiments, the high blood glucose level and the low blood glucose level values are displayed. In other embodiments, as is shown in FIG. 10, the guide lines 1006 are pictorially represented by white lines without values. In some embodiments, a line showing when an insulin dosage should be administered is also be depicted in the graph 1004.

Soft buttons 204 (FIG. 2) display various dosing options in the landing screen 1002. In some embodiments, the soft button 204 information is stored under a patient's preferences 322 (FIG. 3). The patient can choose the “change SETTINGS” options button 1008 to change the settings of his remote device 104 as described in FIG. 8. The patient can choose to initiate an insulin injection by choosing the soft button 204 “dose by CARBS” 1010 or the soft button 204 “dose by UNITS” 1012.

If a patient chooses to “dose by CARBS” 1010, the carbohydrate adjustment screen 1013 appears. In some embodiments, the remote device 104 makes a guess as to how many carbohydrates the patient is likely to eat and displays a suggested carbohydrates amount 1014. This suggested carbohydrate amount 1014 is based on information stored in a patient's profile 328 (FIG. 3). In some embodiments, the patient's profile 328 stores how many carbohydrates a patient guessed he would be eating at all previous meals, the times of those meals, the mode of operation, and the insulin dosage administered. The learning algorithm 320 (FIG. 3) uses the information in the patient profile 328 to determine a suggested carbohydrate amount 1014. The patient can then adjust the number of carbohydrates he or she is planning to eat using the up arrow 1016 or the down arrow 1018. In some embodiments, when the patient is finished, the insulin dosage screen 1022 appears automatically. In other embodiments, the patient verifies that he or she is done making a carbohydrate guess by pressing the “NEXT” key 1020.

The insulin dosage screen 1022 appears after the patient has finished with the carbohydrate adjustment screen 1013. However, the insulin dosage screen 1022 will appear immediately if the patient chooses the “dose by UNITS” 1012 soft button 204 in the landing screen 1002. At the insulin dosage screen 1022, an intelligent guess as to how much insulin is needed is displayed as a suggested insulin dosage 1024. In some embodiments, the amount of insulin left in the patient's body 1026 will also be displayed. In some embodiments, the total amount of insulin that will be in the patient's body after the injection 1028 is also displayed. This additional information may help the patient better understand his or her insulin needs. The patient chooses the up arrow 1016 or the down arrow 1018 to modify the bolus dosage. In some embodiments, when the patient is finished, the dosing verification screen 1030 appears automatically. In other embodiments, the patient chooses to proceed to the dosing verification screen by pressing the “NEXT” key 1020.

The dosing verification screen 1030 displays the amount of insulin to be administered 1032. The patient is then asked to verify that the insulin should be injected now by selecting the “YES” button 1034, or that the insulin should not be injected by selecting the “NO” button 1036. After the patient chooses the “YES” button 1034, the active dosing screen appears 1038 indicating that dosing is currently occurring. The remote controller 104 (FIG. 1) sends the dosage amount to the pump system 106 (FIG. 1), where the correct amount of insulin is then administered to the patient. While the insulin is being injected the patient can monitor the injection progress by observing the progress bar 1040 in the active dosing screen. In some embodiments, after a successful insulin injection has occurred, a success screen appears. In other embodiments, the remote controller 104 immediately returns to the ambient display screen 600.

In some modes 324 (FIG. 3), only a subset of the above described screens appear. For example, the patient can set both the basal and bolus injections to be automatically calculated by the remote device 104 (FIG. 1). In these embodiments, the patient is required to only verify the automatically calculated dosage at the dosing verification screen 1030 prior to active dosing. In still other embodiments, the basal injections can be administered by drip such that no interface screens appear. This might be useful during sleep mode, exercise mode, or a school mode for young children.

In should also be apparent to one skilled in the art, that the above described functions could be accomplished by any similar means. The screens to do not need to look precisely like the ones shown in FIG. 10. For example, in some embodiments, different information can appear on the landing screen 1002. Furthermore, some screens can be combined while the same or similar functions are performed.

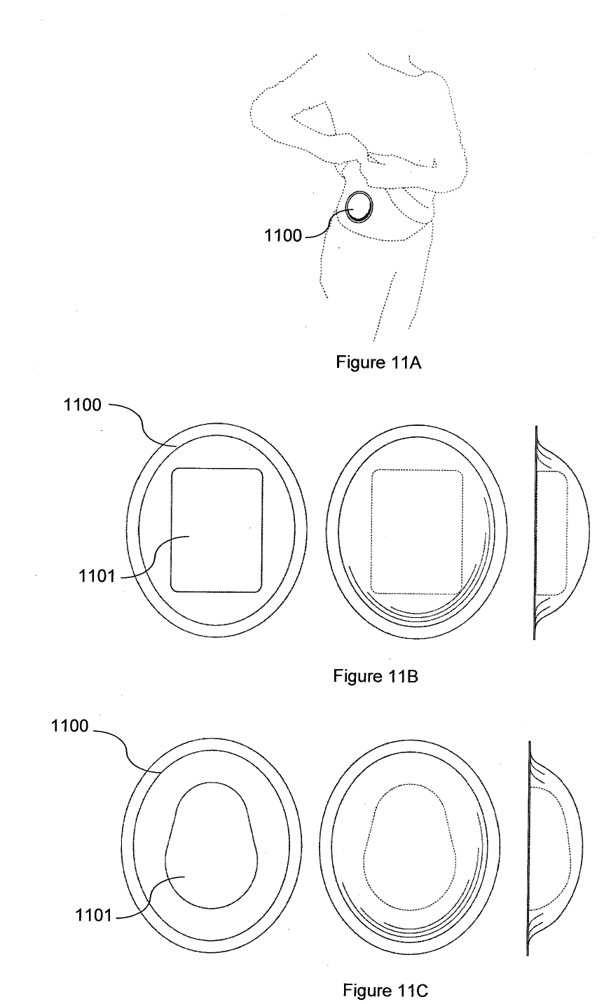

FIG. 11A-C shows an embodiment of a soft shell 100 made of rubber or a rubber-like material. FIG. 11A shows the shell attached to a patient's body. FIGS. 11B and 11C show two possible shapes of the internal cavity 1101 within the shell 1100. The shell 100 can be attached directly to a patient's body in any appropriate place such that the shell firmly attaches to the patient's skin. For example, it can be attached to the patient's stomach, back, side, hip, thigh, etc. The shell 1100 blends in with the patient's body by having a softness, color, and texture similar to the patient's skin. For example, it may be similar to “cutlets”, or breast inserts, that women sometimes wear with strapless dresses. The shell 1100 is configured to smoothly cover the one or more of the following: a glucose monitor system 102, an insulin pump 404, an insulin reservoir 406, a cannula 408, a needle 410, or any other related items shown in FIG. 4. The smoothness of the shell 1100 reduces the likelihood that one or more of these elements will be caught on the patient's clothing or on other external items. Also, the shell allows the patient's clothing to smoothly slide over these elements. As such, these items will not appear bulky or protrude from the patient's clothing, and an external observer may be un-aware that the patient is wearing diabetics related equipment because of the seamless way the shell 1100 blends in with the contours of the patient's body.

In some embodiments, the shell 1100 is attached to the patient by glue around the perimeter of the shell 1100. Alternatively, it can attach through suction or any other suitable attachment means. In some embodiments, the shell 1100 includes a built in insulin reservoir in the internal cavity 1101. In some embodiments, the internal cavity is large enough to cover all of the glucose monitoring system 100 (FIG. 1) items under the shell 1100. In other embodiments, the internal cavity 1101 is configured with individual pockets to fit separately and snugly around the insulin reservoir, the pump, the glucose monitor, or any other bulky piece of the glucose monitoring system 100.

In some embodiments, the shell 1100 is customizable to match the patient's skin tone. In other embodiments, the shell 1100 is clear, brightly colored, patterned, or made to mimic a skin embellishment such as a tattoo, jewel, or cartoon character. In yet other embodiments, the shell 1100 is customizable such that a patient can change its color by swapping out a faceplate or layer. In some embodiments, the shell 1100 is re-usable, while in other embodiments, the shell 1100 is designed for a one time use.

In some embodiments, the shell 1100 is waterproof, and can be worn during swimming or showering. It can be worn for numerous days in a row, and may only need to be removed for maintenance such as to re-fill the insulin reservoir. In some embodiments, the shell 1100 is configured such that the insulin reservoir is refilled by an injection through the shell 1100. In this embodiment, the shell 1100 does not need to be removed for insulin reservoir refilling. A patient can wear the shell 1100 during all normal daily activities such as to work, to school, while exercising, and while sleeping.

The term “patient” has been used throughout this description. However, one skilled in the art would realize that at many times a person other than the patient could be performing the interactions with the remote device. For example, a physician or pharmacist may set the original profile of a patient before the patient uses the device. Also, if the patient was unconscious, another user can interact with the remote device to dose insulin. Alternatively, a patient may be too young or otherwise incapable of understanding how to dose themselves and a parent or guardian may be the one interacting with the remote controller instead.

The foregoing descriptions of specific embodiments of the present invention are presented for purposes of illustration and description. They are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated. Furthermore, the order of steps in the described methods is not necessarily intended to occur in the sequence laid out. In addition, use of singular terms also includes their plural. It is intended that the scope of the invention be defined by the following claims and their equivalents.

What is claimed is:

1. A method for dosing a patient with insulin, comprising:
wirelessly receiving at a remote device a blood glucose level of a patient;
calculating at the remote device an insulin dosage to be administered; and
wirelessly transmitting from the remote device the insulin dosage to be administered to the patient.

2. A method of claim 1, wherein said blood glucose level of a patient is wirelessly received from one or more blood glucose monitoring systems distinct from the remote device, and the insulin dosage to be administered is transmitted to a pump system distinct from the remote device.

3. A method of claim 2, further comprising:
measuring a patient's blood glucose level at the glucose monitoring system attached to a patient;
wirelessly transmitting the patient's blood glucose level from the glucose monitoring system to the remote device;
wirelessly receiving at the pump system from the remote device the insulin dosage to be administered; and
administering insulin corresponding to the insulin dosage to be administered to the patient at the pump system.

4. A method of claim 1, wherein said wirelessly transmitting and said wirelessly receiving are to and from a single pump-monitor system.

5. A method of claim 1, further comprising, prior to said calculating:
determining at the remote device a suggested insulin dosage based on said blood glucose level and a patient profile, which includes historical blood glucose levels, stored on the remote device.

6. A method of claim 5, wherein the insulin dosage to be administered is the suggested insulin dosage.

7. A method of claim 5, wherein determining the insulin dosage to be administered further comprises receiving patient instructions selected from a group consisting of: instructions to modify, accept, or reject the suggested insulin dosage.

8. A method of claim 5, further comprising, prior to said determining at the remote device a suggested insulin dosage:
determining if a dosage cycle trigger level has reached a threshold;
if said dosage cycle trigger level has reached a threshold, initiating a dosage cycle;
if said dosage cycle trigger level has not reached a threshold, performing no further operations.

9. A method of claim 8, wherein the pre-determine dosage cycle trigger level is selected from the group consisting of: a blood glucose level, a time of day, a period of time since a previous insulin dosage, and an alert message.

10. A method of claim 1, further comprising, prior to said calculating:
receiving from a patient an instruction to begin said calculating.

11. A method of claim 10, wherein said patient instructions are selected from the group consisting of: an instruction to begin an automatic dosage calculation, patient inputted insulin dose, and patient inputted units of carbohydrates.

12. A method of claim 10, wherein said instruction includes a patient desired insulin dosage, further comprising, prior to said wirelessly transmitting:
comparing said patient desired insulin dosage to said insulin dosage to be administered and obtaining a difference; and
if said difference reaches a pre-determined warning trigger level, returning a patient specific warning;
if said difference does not reach a predetermined warning trigger level, displaying said patient desired insulin dosage as a calculated insulin dosage to be administered.

13. A method of claim 1, further comprising, prior to said calculating:
comparing the blood glucose level to a previous blood glucose level; and
displaying a blood glucose level trending data in a format selected from a group consisting of: a trending arrow, a trending graph, a change in an ambient display, and any combination of the aforementioned.

14. A method of monitoring a patient's blood glucose levels, comprising:
receiving at a remote device a blood glucose level of a patient;
comparing the blood glucose level to one or more previous blood glucose levels of the patient; and
displaying a blood glucose level trending data as a change in an ambient display.

15. A method of claim 14, wherein the change in ambient display presents a positive display image when trending in a normal range and a negative display image when trending outside of, or nearly outside of, the normal range.

16. A method of claim 15, wherein the positive and negative displays are selected from a group consisting of: a darker sky when trending outside of the normal range and a lighter sky when trending in the normal range, an unhappy digital pet when trending outside of the normal range and a happy digital pet when trending in the normal range, a warm color when trending outside of the normal range and a cool color when trending in the normal range, and any combination of the aforementioned.

17. A method of claim 14, further comprising:
storing the patient's blood glucose level in a patient profile along with one or more of the following pieces of information: time, date, and mode, insulin dose, previous carbohydrate consumption guess, the amount of the previous dose, the time since the previous dose, and any warnings or alerts that were given.

18. A method of claim 1, wherein said wirelessly receiving activates an alert if the blood glucose level reaches below a predetermined blood glucose alert level, where said alert is in a format selected from a group consisting of: a visual alert, a haptic alert, an audio alert, and any combination of the aforementioned.

19. A method of claim 1, wherein said wirelessly receiving activates an alert if a technical problem occurs; said technical problem is selected from a group consisting of: a low insulin level in a reservoir, a malfunction in the blood glucose monitoring system, a malfunction in the pump system, the pump is disconnected from a cannula, a cannula is disconnected from a needle, the remote device is out of range with the blood glucose monitoring system, and the remote device is out of range with the pump system.

20. A method of claim 5, wherein said suggesting is at least partially based on a patient specified mode selected from the group consisting of: a sleep mode, a rest mode, an exercise mode, a work mode, a school mode, an eating mode, and a default mode.

21. A method of claim 1, further comprising,
sending a patient profile to an external computing device for tracking long term trends, tracking trends in various modes, or assisting patients in goal setting.

22. A diabetes management method, said method comprising:
measuring a patient's blood glucose level at a glucose monitoring system attached to a patient;
wirelessly transmitting the patient's blood glucose level from the glucose monitoring system to a remote device;
wirelessly receiving at the remote device the blood glucose level of the patient;
comparing the blood glucose level to a previous blood glucose level;
displaying a blood glucose level trending data on the remote device;
simultaneously displaying the blood glucose level on the remote device;
comparing the blood glucose level to a pre-determined dosage cycle trigger level;
determining whether to initiate a suggested dosage cycle based on said comparing;
determining at the remote device a suggested insulin dosage based on the blood glucose level and a patient profile, which includes historical blood glucose levels, stored on the remote device;
calculating at the remote device an insulin dosage to be administered;
receiving instructions from the patient to transmit said insulin dosage to be administered;
wirelessly transmitting from the remote device to a pump system attached to a patient the insulin dosage to be administered;
wirelessly receiving at the pump system from the remote device the insulin dosage to be administered; and
administering insulin corresponding to the insulin dosage to be administered to the patient at the pump system.
